(19) United States
(12) Patent Application Publication
(10) Pub. No.: US 2023/0049597 A1
(43) Pub. Date: Feb. 16, 2023

(54) DEVICES AND METHODS FOR AUDITORY REHABILITATION FOR INTERAURAL ASYMMETRY

(71) Applicant: DEBORAH MONCRIEFF, MEMPHIS, TN (US)

(72) Inventor: DEBORAH MONCRIEFF, MEMPHIS, TN (US)

(21) Appl. No.: 17/976,832

(22) Filed: Oct. 30, 2022

Related U.S. Application Data
(63) Continuation of application No. PCT/US21/30499, filed on May 3, 2021.
(60) Provisional application No. 63/018,868, filed on May 1, 2020.

(51) Int. Cl. A61F 11/04; G10L 15/22; G10L 25/66; H04R 1/10; G10L 21/0364; H04S 1/00; H04S 7/00; A61F 11/06

(52) U.S. Cl. CPC A61F 11/04; G10L 15/22; G10L 25/66; H04R 1/1083; G10L 21/0364; H04R 1/1041; H04S 1/007; H04S 7/30; A61F 11/06; H04R 2460/01; H04S 2400/15; H04S 2420/01

(57) ABSTRACT

A device, system and related methods to provide assessment and treatment of amblyaudia through standardized methods that do not require advanced training or a booth with loudspeakers for the operator to administer. The ARIA stimuli protocols for both assessment and treatment, encoded in or to be used by a software program or application, are transferred to a stand-alone set of specialized noise-cancelling headphones attached or connected to, wired or wirelessly, a software platform on an electronic computing device. or integrated with the headphones. The program administers assessment tests to individuals through the noise-cancelling earphones. The device enables someone with minimal instructions to administer automatically or semi-automatically both assessment and treatment protocols, generate results, make interpretations, store data, and produce reports. The device or system may be loaded with standard protocols for English-speaking individuals, as well as dichotic speech material in any language.

This application is a continuation of PCT/US21/30499, filed May 3, 2021 by Deborah Moncrieff, entitled “DEVICES AND METHODS FOR AUDITORY REHABILITATION FOR INTERAURAL ASYMMETRY,” which claims priority to and benefit of U.S. Provisional App. No. 63/018,868, filed May 1, 2020, the complete disclosures of both of which, including all appendices, are incorporated herein by specific reference for all purposes.

### FIELD OF INVENTION

This invention relates to an apparatus, system, and related methods for the diagnosis and treatment of amblyaudia, more particularly to remediate the interaural asymmetry that is the hallmark of amblyaudia through Auditory Rehabilitation for Interaural Asymmetry auditory protocols.

### BACKGROUND OF INVENTION

Amblyaudia is a deficit in the binaural integration of sounds from the environment that has a negative impact on listening, learning, communication, and academic performance. Amblyaudia occurs in 15% to 20% of school-age children, and if left untreated, persists into adulthood with a range of likely deleterious effects.

Binaural integration is the cognitive process that involves the combination of different auditory information presented binaurally (e.g., the ability to process different information being present to both ears simultaneously). The brainstem uses timing and intensity difference to help integrate the signals. Interaural time differences result when signals from each side of the head arrive at the ear on the nearer side of the head earlier, and later at the opposite ear. Interaural intensity differences results when the intensity for signals arriving from one side are reduced at the opposite ear, typically resulting from a shadow effect created by the head and/or torso. Intensity differences are more significant for higher frequency signals. Binaural integration thus helps with localization (i.e., direction and distance of a signal source) and understanding speech in background noise.

Accordingly, what is needed is device and related methods for the diagnosis and automated treatment of amblyaudia.

### SUMMARY OF INVENTION

In various embodiments, the present invention comprises a device or system and related methods to provide assessment and treatment of amblyaudia through standardized methods that do not require advanced training or a booth with loudspeakers for the operator to administer. The ARIA stimuli protocols for both assessment and treatment, encoded in or to be used by a software program or application, are transferred to a stand-alone set of specialized noise-cancelling headphones attached or connected to, wired or wirelessly, a software platform on an electronic computing device, such as a computer, laptop, mobile computing device, tablet, or notepad, or integrated with the headphones. The software program administers assessment tests to individuals through the noise-cancelling earphones. The device enables someone with minimal instructions to administer automatically or semi-automatically both assessment and treatment protocols, generate results, make interpretations, store data, and produce reports. The device or system may be loaded with standard protocols for English-speaking individuals, as well as subsequently developed dichotic speech material in any language, thereby enabling individuals in any country to benefit from the assessment and treatment of this common listening problem.

### BRIEF DESCRIPTION OF THE DRAWINGS

FIG. 1 shows a diagram of a system for amblyaudia detection and treatment in accordance with an exemplary embodiment of the present invention.

FIG. 2 shows a conceptual comparison of dichotic listening tests to monotic and diotic listening tests.

FIG. 3 shows a diagram of headphones delivering sounds with modified intensity.

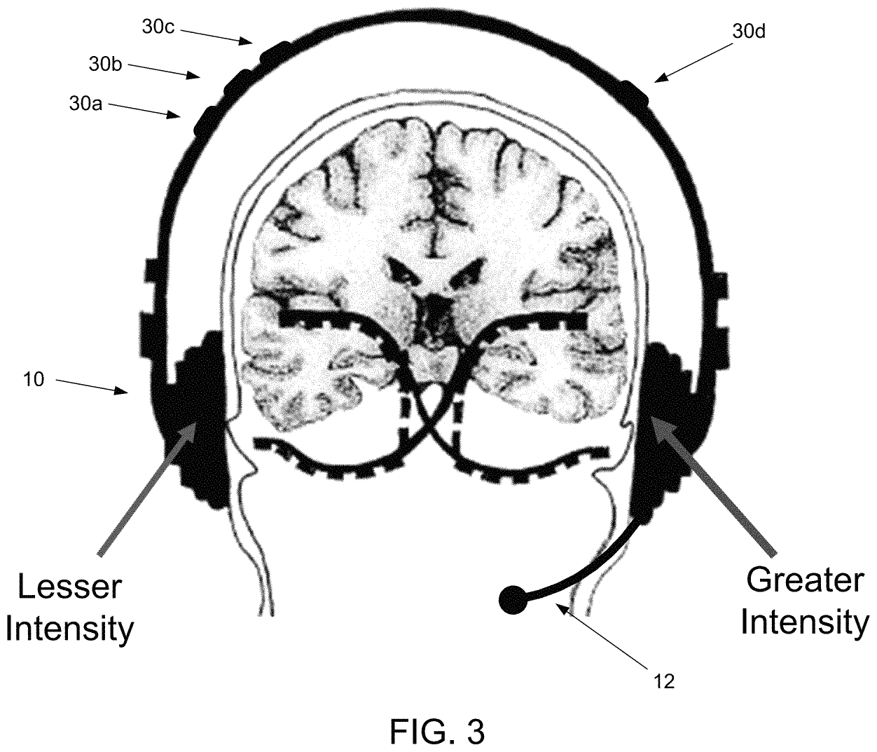

FIGS. 4A-B show representative diagrams of how the signals to the left (L) and right (R) ears of a subject are modified to replicate an interaural time delay and an interaural level difference experienced by a hypothetical subject listening to a hypothetical sound source in the sound produced by an output device (e.g., earphones) of the present invention.

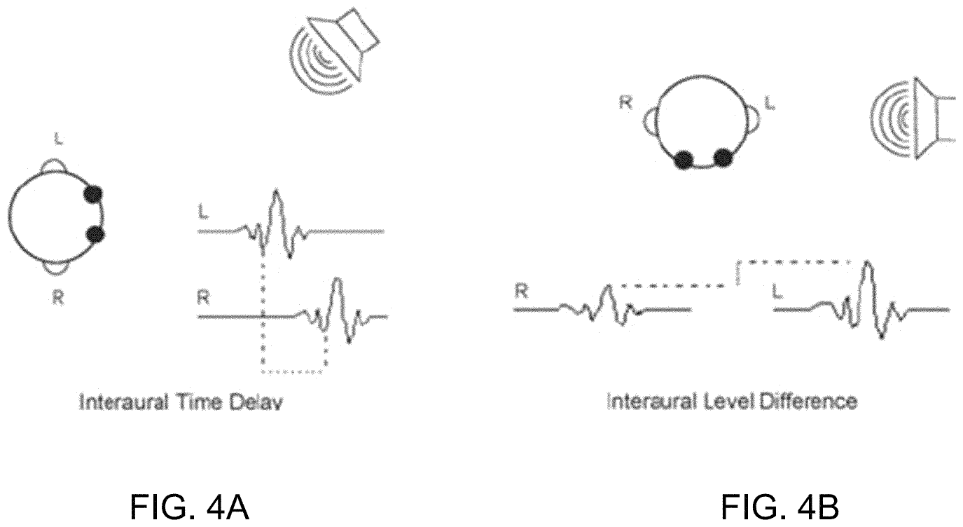

### DETAILED DESCRIPTION OF EXEMPLARY EMBODIMENTS

In various exemplary embodiments, the present invention comprises an apparatus, system, and related methods for the diagnosis and treatment of amblyaudia. Implementation of widespread testing and treatment for amblyaudia can potentially improve academic outcomes in schoolchildren, listening and communication in people of all ages, and enhanced benefits from amplification devices for individuals with hearing loss. Devices in accordance with the present invention provide assessment and treatment in multiple languages for use with English and/or non-English speaking individuals in the U.S. and other countries around the world.

Auditory Rehabilitation for Interaural Asymmetry (ARIA) is a short-term (e.g., 4 one-hour sessions) auditory protocol designed to remediate the interaural asymmetry that is the hallmark of amblyaudia (i.e., one non-dominant ear is significantly poorer in binaural integration than the other, dominant ear). The protocol is grounded in the principles of neuroplasticity induced through active potentiation of neural connections responsible for binaural processing of sounds from the environment. In general, ARIA provides strong stimulation to the weaker, non-dominant side while inhibiting or constraining the input to the dominant side, thereby producing synaptic neuroplasticity that facilitates aural processing through the weaker side or channel. ARIA reduces interaural asymmetry, and benefits remain stable with only minimal regression over several years.

The treatment follows a standardized protocol that requires advanced training and preparation for clinicians to use. The current methods for clinician-driven training are conducted in sound-treated booths with stimuli delivered from free-field speakers, thereby involving the natural head-related transfer function that occurs in daily listening. Stimuli are presented in the sound field, effectively matching daily listening situations, with a natural binaural advantage of HRTF due to the free-field speakers. However, clinicians must be trained to closely monitor and record patient/subject responses and adjust interaural intensities according to the standardized protocol to achieve maximum benefit.

This level of training, and the apparatus necessary, thus limit ARIA therapy to audiology clinical sites, and limits the implementation and use of ARIA. The present invention is a significant improvement in ARIA therapy, eliminating the size and type of apparatus necessary as well as the need for clinicians with advanced training.

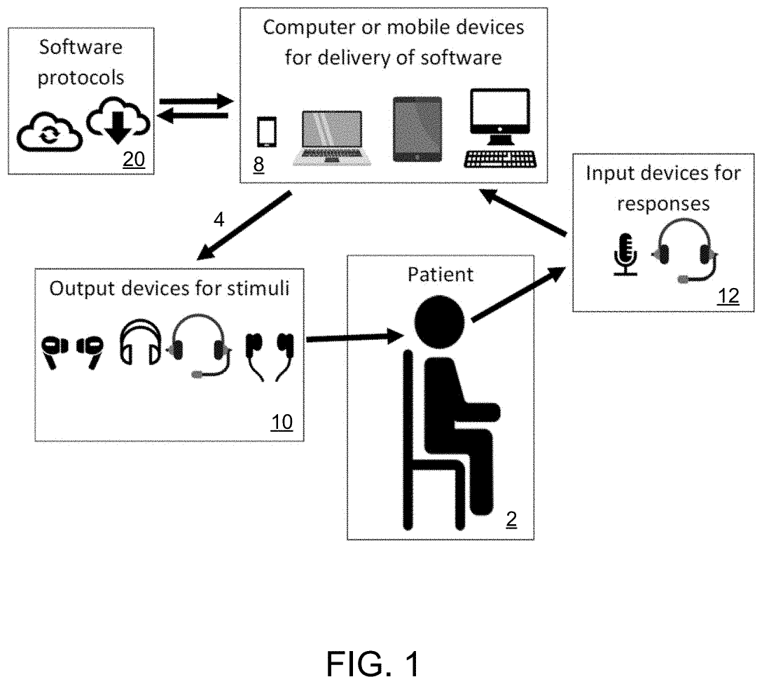

In the present invention, as seen in FIG. 1, the patient/subject 2 listens to the ARIA sound stimuli 4 received from a computer or mobile computing device (such as, but not limited to, a computer, laptop, mobile computing device, tablet, or notepad) 8, programmed to generate and/or process the sound stimuli based on protocols code in or used by software 20, through output devices 10 worn in or on both of the ears, such as, but not limited to, earphones, earbuds, headphones a headset with microphone, or other similar hearing devices, wired or wireless (generally referred to hereafter as “headphones” or “earphone”). In a preferred embodiment, the headphones should be configured to cover the ears (e.g., padded ear cuffs) or be inserted into the outer ear canal in such a way as to reduce interference from ambient noise or sound. If inserted into the outer ear canal, the earbuds or the like may be contoured to fit the ear, and may comprise different sizes and shapes. The present invention thus eliminates the need for a sound-proof booth with free-field speakers.

Because the natural interaural differences in timing and intensity/level that result from the prior art booth apparatus are no longer present, the timing and intensity/level differences are replicated in the sound stimuli presented in the headphones. More specifically, the natural head-related transfer function is replicated through modifications to the ARIA sound stimuli for delivery through the ear-level headphones.

The patient provides a spoken response to the sound stimuli, which is received by a voice input device 12, such as, but not limited to, a standalone microphone or a microphone integrated with output devices (e.g., a headset with a microphone). The voice input device transmits, wired or wirelessly, the response to a computer device 8 (which may be the same computer or mobile computing device programmed to generate/process the sound stimuli).

One or more programmed applications 20 loaded in computer memory on the computer or mobile computing device are programmed to use voice-activation principles to monitor the patient responses, and to make automatic adjustments to subsequent stimulus intensities and/or timing based on the patient/subject's responses. The present invention thus also eliminates the advanced training currently required for a clinician to deliver ARIA treatment, and allows an individual to be assessed for amblyaudia and administered ARIA with minimal instructions at any quiet location.

In one embodiment, a device or system in accordance with the present invention delivers assessment and treatment of amblyaudia through standardized methods that do not require advanced training or a booth with loudspeakers for the operator to administer. To achieve this aim, the stimuli protocols for both assessment and treatment, encoded in or to be used by a software program or application 20, are transferred to a stand-alone set of specialized noise-cancelling headphones 10 attached or connected, wired or wirelessly, to a software platform on an electronic computing device 8, such as a computer, laptop, mobile computing device, tablet, or notepad. A software program loaded on the computing device administers assessment tests to individuals through the noise-cancelling earphones, which are attached or connected in a similar fashion. The device enables someone with minimal instructions to administer automatically or semi-automatically both assessment and treatment protocols, generate results, make interpretations, store data, and produce reports. The device or system may be loaded with standard protocols for English-speaking individuals, as well as subsequently developed dichotic speech material in any language, thereby enabling individuals in any country to benefit from the assessment and treatment of this common listening problem.

In additional embodiments, the present invention converts treatment sound files to head-related transfer function (HRTF) versions to emulate current clinical standards whereby the material is delivered free-field without headphones. Sound files of dichotic speech material (which comprise two or more sound sources delivered to both ears) used in the clinical ARIA protocol are modified to emulate the head-related transfer function (HRTF) that occurs when speech material is delivered from a sound source located three feet from the person's ear(s). In one embodiment, the system uses a KEMAR-generated electronic filter applied to the standard HRTF sound files to create modified sound files for presentation (KEMAR is the Knowles Electronic Manikin for Acoustic Research, a head-and-torso simulator commonly used for testing and research of hearing and acoustic devices). Once modified, the temporal and spectral features of the sounds are delivered to each earphone as if the person were sitting in the sound field not wearing earphones. Each earphone channel thus may receive direct input of the “same side” signal (i.e., the signal as it would be received in a listener's ear closest to a natural sound field environmental sound source) and/or indirect input (e.g., delayed and/or less intense) of the “opposite side” signal (i.e., the signal as it would be received in the listener's ear further from the environmental sound source).

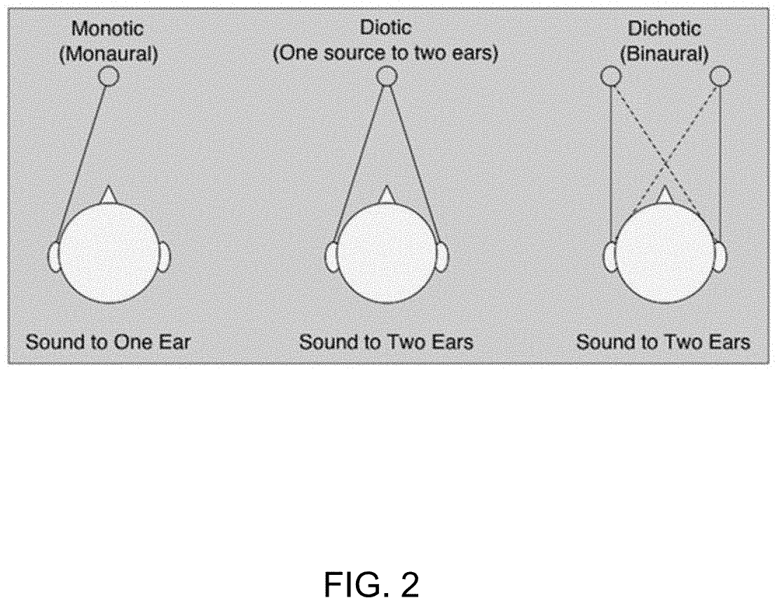

Thus, for example, the sound intensity delivered to one ear is higher (or lower) than the intensity of that sound delivered to the other ear. Similarly, the timing of the sound delivered to one ear is earlier (or later) than the timing of that sound delivered to the other ear. This allows the same sound (e.g., a word) to be delivered with different intensity and/or timing modifications to the non-dominant ear and the dominant ear, as seen in FIG. 2. In some cases, just intensity or timing is modified, while in other cases, both intensity and timing are modified. This produces a “virtual” listening experience that more closely matches naturalistic listening, and matches the delivery of ARIA therapy done in the sound field in a booth with speakers.

The software program directs the sequence of the training protocol according to standardized methods. The program delivers the sound files, tracks listener responses, and directs changes in intensity (and/or timing) of the material being presented to one of the two ears throughout the training process.

While FIG. 1 shows the computing device as separate from the headphones, the computing device components may be integrated with and contained in the headphones. Accordingly, in one embodiment, the headphones may comprise one or more CPUs and/or microprocessors 30a, computer memory 30b, electronic computer storage 30c, and electronic communications devices 30d (e.g., wired and/or wireless), that are capable of receiving, processing, storing, modifying, operating and using the above-described sound files of dichotic speech material and associated software program(s). These components may be located in the earpieces, on a headband or apparatus connecting the earpieces, or a combination thereof. The headphones may further comprise at least one integrated voice input device 12, such as a microphone as described elsewhere herein, that receives the vocal responses from the user, and sends those responses to the software program operating on the headphones for processing as described above. The headphones with integrated computing device components may be in the form of a standard set of headphones, or may be presented as a hat, helmet or other form of headgear.

In several embodiments, voice-recognition is used to score listener results and use those results to direct intensity changes to the presentation of material to one ear. For example, the material presented comprises a list of spoken words, with the intensity and/or timing of the delivery of each word modified as described above. The listener repeats the word after hearing the word. The program records the listener's responses and scores them as correct or incorrect. The scoring may be performed after each word or after each word list. The word lists used for both assessments and treatment are a closed set so voice-recognition will be relatively straightforward.

Headphones are selected that have appropriate properties to deliver standard format for assessment (i.e., simple simultaneous delivery of one word to one earphone and another word to the other earphone), and to deliver speech material recorded onto binaural tracks with the HRTF modifications needed for therapy (i.e., each word to one earphone that is also present in the other earphone at lower intensity and/or later in time as if the listener is in the sound field). Headphones are evaluated with calibration and spectral analysis methods to validate that the stimuli are delivered appropriately. Headphones may be equipped with either a boom or integrated microphone that adequately records the listener's responses and allows the software program to score them and compare them at the end of each word list.

During the assessment phase, the present invention scores the responses and compares them to stored normative responses (which may be specific by age, gender, stimulus type, or other characteristics). It then interprets the results to determine whether the responses were normal or abnormal. Results are stored in a database (typically at each implementation site), which can then be shared across multiple sites for research. In the training or treatment phase, the system will score responses for each word list presented, compare the scores in the two ears, and then follow a prescribed algorithm to make the systematic adjustments to the intensity of the information presented to the listener's dominant ear. The therapy maintains performance in the non-dominant ear while systematically adjusting intensity in the dominant ear until excess interaural asymmetry is no longer present when the two ears are hearing speech at the same intensity level.

Accordingly, the present invention possesses several advantages over the prior art. Current testing and treatment for amblyaudia is performed by a clinician in a sound-treated booth at an audiologic clinical practice, severely limiting access to individuals with health insurance coverage for diagnostics and in many cases, financial resources to private-pay for the treatment. Given the widespread prevalence of amblyaudia, this means that a vast majority of children with this binaural integration deficit are undiagnosed. Even with widespread testing and diagnosis, however, the current clinician-driven treatment is available in only a handful of locations and is rarely covered by health insurance. The present invention with its automatic and/or semi-automatic operation dramatically increases the availability and ease of both diagnosis and treatment, and provides important benefits to individuals of all ages who may suffer from this listening problem. With portability and ease of administration, devices in accordance with the present invention can be disseminated and implemented for a wide range of individuals of all ages. The device's plans for delivery of stimuli via noise-cancelling binaural earphones can be adapted to include delivery via Bluetooth to earbuds, amplification devices, and sound-field speakers for use with a wider variety of individuals. Similarly, the device's plans for feedback via voice-activation can be adapted to include feedback via hand-held devices, computers and augmentative, alternative communication devices for populations with special needs. Another advantage to the proposed device is the inclusion of a privacy-compliant database for storage of information on assessment, treatment protocols, and patient outcomes for dissemination in the research community.

Devices in accordance with the present invention can be used by teachers, school administrators, audiologists, speech-language pathologists, nurses, physicians, psychologists, parents, and/or family members wishing to test an individual with listening, language, and communication difficulties to detect and confirm the presence of amblyaudia, and if present, administer automatic or semi-automatic ARIA treatment for it. A device may be simple or may include additional enhancements as described herein. The device is programmed to automatically drive the protocols for assessment and treatment, and to automatically adjust stimulus output based on scoring patient/subject responses. All additional or supplemental stimuli may be modified with the head-related transfer function to simulate naturalistic listening, and reprogrammed for delivery through noise-cancelling earphones, or other embodiments of the present invention.

These embodiments, as well as other exemplary embodiments, as well as the tools and programs referenced above, and background information about ARIA, are described in detail in the appendices to U.S. Provisional App. No. 63/018,868, filed May 1, 2020 which is incorporated herein in its entirety (including all text, figures, and appendices) by specific reference for all purposes.

In order to provide a context for the various computer-implemented aspects of the invention, the following discussion provides a brief, general description of a suitable computing environment in which the various aspects of the present invention may be implemented. A computing system environment is one example of a suitable computing environment, but is not intended to suggest any limitation as to the scope of use or functionality of the invention. A computing environment may contain any one or combination of components discussed below, and may contain additional components, or some of the illustrated components may be absent. Various embodiments of the invention are operational with numerous general purpose or special purpose computing systems, environments or configurations. Examples of computing systems, environments, or configurations that may be suitable for use with various embodiments of the invention include, but are not limited to, personal computers, laptop computers, computer servers, computer notebooks, hand-held devices, microprocessor-based systems, multiprocessor systems, TV set-top boxes and devices, programmable consumer electronics, cell phones, personal digital assistants (PDAs), tablets, smart phones, touch screen devices, smart TV, internet enabled appliances, internet enabled security systems, internet enabled gaming systems, internet enabled watches; internet enabled cars (or transportation), network PCs, minicomputers, mainframe computers, embedded systems, virtual systems, distributed computing environments, streaming environments, volatile environments, and the like.

Embodiments of the invention may be implemented in the form of computer-executable instructions, such as program code or program modules, being executed by a computer, virtual computer, or computing device. Program code or modules may include programs, objects, components, data elements and structures, routines, subroutines, functions and the like. These are used to perform or implement particular tasks or functions. Embodiments of the invention also may be implemented in distributed computing environments. In such environments, tasks are performed by remote processing devices linked via a communications network or other data transmission medium, and data and program code or modules may be located in both local and remote computer storage media including memory storage devices such as, but not limited to, hard drives, solid state drives (SSD), flash drives, USB drives, optical drives, and internet-based storage (e.g., “cloud” storage).

In one embodiment, a computer system comprises multiple client devices in communication with one or more server devices through or over a network, although in some cases no server device is used. In various embodiments, the network may comprise the Internet, an intranet, Wide Area Network (WAN), or Local Area Network (LAN). It should be noted that many of the methods of the present invention are operable within a single computing device.

A client device may be any type of processor-based platform that is connected to a network and that interacts with one or more application programs. The client devices each comprise a computer-readable medium in the form of volatile and/or nonvolatile memory such as read only memory (ROM) and random access memory (RAM) in communication with a processor. The processor executes computer-executable program instructions stored in memory. Examples of such processors include, but are not limited to, microprocessors, ASICs, and the like.

Client devices may further comprise computer-readable media in communication with the processor, said media storing program code, modules and instructions that, when executed by the processor, cause the processor to execute the program and perform the steps described herein. Computer readable media can be any available media that can be accessed by computer or computing device and includes both volatile and nonvolatile media, and removable and non-removable media. Computer-readable media may further comprise computer storage media and communication media. Computer storage media comprises media for storage of information, such as computer readable instructions, data, data structures, or program code or modules. Examples of computer-readable media include, but are not limited to, any electronic, optical, magnetic, or other storage or transmission device, a floppy disk, hard disk drive, CD-ROM, DVD, magnetic disk, memory chip, ROM, RAM, EEPROM, flash memory or other memory technology, an ASIC, a configured processor, CDROM, DVD or other optical disk storage, magnetic cassettes, magnetic tape, magnetic disk storage or other magnetic storage devices, or any other medium from which a computer processor can read instructions or that can store desired information. Communication media comprises media that may transmit or carry instructions to a computer, including, but not limited to, a router, private or public network, wired network, direct wired connection, wireless network, other wireless media (such as acoustic, RF, infrared, or the like) or other transmission device or channel. This may include computer readable instructions, data structures, program modules or other data in a modulated data signal such as a carrier wave or other transport mechanism. Said transmission may be wired, wireless, or both. Combinations of any of the above should also be included within the scope of computer readable media. The instructions may comprise code from any computer-programming language, including, for example, C, C++, C#, Visual Basic, Java, and the like.

Components of a general purpose client or computing device may further include a system bus that connects various system components, including the memory and processor. A system bus may be any of several types of bus structures, including, but not limited to, a memory bus or memory controller, a peripheral bus, and a local bus using any of a variety of bus architectures. Such architectures include, but are not limited to, Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computing and client devices also may include a basic input/output system (BIOS), which contains the basic routines that help to transfer information between elements within a computer, such as during start-up. BIOS typically is stored in ROM. In contrast, RAM typically contains data or program code or modules that are accessible to or presently being operated on by processor, such as, but not limited to, the operating system, application program, and data.

Client devices also may comprise a variety of other internal or external components, such as a monitor or display, a keyboard, a mouse, a trackball, a pointing device, touch pad, microphone, joystick, satellite dish, scanner, a disk drive, a CD-ROM or DVD drive, or other input or output devices. These and other devices are typically connected to the processor through a user input interface coupled to the system bus, but may be connected by other interface and bus structures, such as a parallel port, serial port, game port or a universal serial bus (USB). A monitor or other type of display device is typically connected to the system bus via a video interface. In addition to the monitor, client devices may also include other peripheral output devices such as speakers and printer, which may be connected through an output peripheral interface.

Client devices may operate on any operating system capable of supporting an application of the type disclosed herein. Client devices also may support a browser or browser-enabled application. Examples of client devices include, but are not limited to, personal computers, laptop computers, personal digital assistants, computer notebooks, hand-held devices, cellular phones, mobile phones, smart phones, pagers, digital tablets, Internet appliances, and other processor-based devices. Users may communicate with each other, and with other systems, networks, and devices, over the network through the respective client devices.

Thus, it should be understood that the embodiments and examples described herein have been chosen and described in order to best illustrate the principles of the invention and its practical applications to thereby enable one of ordinary skill in the art to best utilize the invention in various embodiments and with various modifications as are suited for particular uses contemplated. Even though specific embodiments of this invention have been described, they are not to be taken as exhaustive. There are several variations that will be apparent to those skilled in the art.

What is claimed is:

1. A system for assessing and treating amblyaudia, comprising:
a sound output device configured to be worn on the head of a user, wherein the sound output device covers or is inserted into both ears of the user;
a computing device comprising a microprocessor in electronic communication with a computer memory and a computer digital storage device;
a voice input device configured to receive oral input from the user; and
one or more computer program applications loaded in said computer memory and operable to process auditory rehabilitation for interaural asymmetry (ARIA) files, and electronically reproduce one of said ARIA files through said sound output device;
wherein the voice input device is configured to receive a vocal response from the user in response to electronic reproduction of at least a portion of the transmitted ARIA file, and to transmit said vocal response to the one or more computer program applications;
further wherein the one or more computer program applications are operable to modify a second of said ARIA files based at least in part on said transmitted vocal response.

2. The system of claim 1, wherein the sound output device is a pair of earphones, a pair of headphones, or a pair of earbuds.

3. The system of claim 1, wherein the sound output device is noise-cancelling.

4. The system of claim 1, wherein the voice input device comprises a microphone.

5. The system of claim 1, wherein the voice input device is integrated with the sound output device.

6. The system of claim 1, wherein the computing device is integrated with the sound output device.

7. The system of claim 1, wherein the voice input device and the computing device are integrated with the sound output device.

8. The system of claim 1, further wherein the second ARIA file is modified by changing the intensity level and/or timing delay in the signal to one ear of the user through the sound output device.

9. The system of claim 1, further wherein the second ARIA file is modified only after a plurality of vocal responses have been received from the user.

10. The system of claim 1, wherein the user has a dominant ear and a non-dominant ear, and the second ARIA file is modified by increasing the intensity level in the signal to the non-dominant ear of the user through sound output device.

11. The system of claim 1, wherein the one or more ARIA files comprise dichotic speech material.

12. The system of claim 1, wherein the one or more ARIA files are converted from free-field treatment sound files to modified head-related transfer function versions for use with the sound output device.

13. The system of claim 12, wherein the one or more ARIA files comprise speech material recorded onto binaural tracks.

14. The system of claim 1, wherein the one or more ARIA files comprise amblyaudia diagnosis files, configured to determine whether the user has amblyaudia.

15. The system of claim 1, wherein the one or more ARIA files comprise amblyaudia treatment files.
